(19) United States
(12) Patent Application Publication
(10) Pub. No.: US 2010/0248322 A1
(43) Pub. Date: Sep. 30, 2010

(54) CHEMICAL AMENDMENTS FOR THE STIMULATION OF BIOGENIC GAS GENERATION IN DEPOSITS OF CARBONACEOUS MATERIAL

(75) Inventors: Robert S. Pfeiffer, Parker, CO (US); Glenn A. Ulrich, Golden, CO (US); Shelley Haveman, Lakewood, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LUCA Technologies, Inc., Golden, CO (US)

(21) Appl. No.: 12/782,311

(22) Filed: May 18, 2010

Related U.S. Application Data
(63) Continuation-in-part of application No. 12/751,745, filed on Mar. 31, 2010.
Application No. 12/751,745 is a division of application No. 11/399,099, filed on Apr. 5, 2006, now Pat. No. 7,696,132.

(51) Int. Cl. C12P 5/02; C12P 1/00; C12P 3/00

(52) U.S. Cl. 435/167; 435/41; 435/168

(57) ABSTRACT

Methods of stimulating biogenic production of a metabolic product with enhanced hydrogen content are described. The methods may include accessing a geologic formation that includes carbonaceous material. The methods may also include introducing an amendment to the geologic formation. The amendments may further include removing compounds with enhanced hydrogen content from the formation.

### CROSS-REFERENCES TO RELATED APPLICATIONS

This application is a continuation-in-part of U.S. patent application Ser. No. 12/751,745, filed Mar. 31, 2010, which is a divisional of U.S. patent application Ser. No. 11,399,099 (now U.S. Pat. No. 7,696,132) filed Apr. 5, 2006, the entire disclosures of which are herein incorporated by reference for all purposes.

### BACKGROUND OF THE INVENTION

Increasing world energy demand is creating unprecedented challenges for recovering energy resources, and mitigating the environmental impact of using those resources. Some have argued that the worldwide production rates for oil and domestic natural gas will peak within a decade or less. Once this peak is reached, primary recovery of oil and domestic natural gas will start to decline, as the most easily recoverable energy stocks start to dry up. Historically, old oil fields and coal mines are abandoned once the easily recoverable materials are extracted. These abandoned reservoirs, however, still contain significant amounts of carbonaceous material. The Powder River Basin in northeastern Wyoming, for example, is still estimated to contain approximately 1,300 billion short tons of coal. Just 1% of the Basin's remaining coal converted to natural gas could supply the current annual natural gas needs of the United States (i.e., about 23 trillion cubic feet) for the next four years. Several more abandoned coal and oil reservoirs of this magnitude are present in the United States.

As worldwide energy prices continue to rise, it may become economically viable to extract additional oil and coal from these formations with conventional drilling and mining techniques. However, a point will be reached where more energy has to be used to recover the resources than can be gained by the recovery. At that point, traditional recovery mechanisms will become uneconomical, regardless of the price of energy. Thus, new recovery techniques are needed that can extract resources from these formations with significantly lower expenditures of energy and costs.

One route for light hydrocarbon recovery that has received little commercial attention is biogenic conversion of carbonaceous materials in geologic formations into methane. As noted above, large potential sources of methane and other hydrocarbons with enhanced hydrogen content are locked up in the carbonaceous materials in coal, residual oil, etc. In biogenic conversion, microorganisms in the formation treat these carbonaceous materials as a food source and metabolize them into metabolic intermediates and products, such as alcohols, organic acids, aromatic compounds, hydrogen and methane, among others.

In many formations, however, the environmental chemistry does not favor the biogenic production of metabolic products like hydrogen and methane. In some of these formations, the presence of an inhibitor (e.g., saline) can prevent the microorganisms from metabolizing the carbonaceous substrate into the products. In other formations, the low concentration of one or more compounds (e.g., nutrient compounds) in the formation environment can slow or stop biogenic production of the products. In still other formations, a rise in concentration of a metabolic intermediate or product generated by an active consortium of microorganisms can slow additional metabolic activity.

Thus, there remains a need to identify chemical compounds that effect the rate of biogenic production of metabolic products by microorganisms in a formation environment. There also remains a need for methods of introducing chemical amendments to a geologic formation that will stimulate the biogenic production of the metabolic products. These and other needs are addressed by the present invention.

### BRIEF SUMMARY OF THE INVENTION

Embodiments of the invention include methods of stimulating biogenic production of a metabolic product with enhanced hydrogen content. The methods may include accessing a consortium of microorganisms in a geologic formation that includes a carbonaceous material. The methods may also include providing hydrogen and one or more phosphorous compounds to the microorganisms. The combination of the hydrogen and phosphorous compound stimulates the consortium to metabolize the carbonaceous material into a metabolic product with enhanced hydrogen content.

Embodiments of the invention also include additional methods of stimulating biogenic production of a metabolic product with enhanced hydrogen content. The methods may include accessing a consortium of microorganisms in a geologic formation that includes a carbonaceous material and providing a carboxylate compound to the microorganisms. The carboxylate compound stimulates the consortium to metabolize carbonaceous material in the formation into the metabolic product with enhanced hydrogen content.

Embodiments of the invention still also include methods of activating a consortium of microorganisms in a geologic formation to produce a metabolic product with enhanced hydrogen content. The methods may include accessing the consortium in the formation, and providing an acetate compound to the microorganisms. The acetate compound activates the consortium to metabolize carbonaceous material in the formation into the metabolic product with enhanced hydrogen content.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

FIG. 1 is a flowchart illustrating a method of introducing hydrogen and phosphorous amendment to microorganisms in geologic formations according to embodiments of the invention;

FIG. 2 is a flowchart illustrating a method of introducing carboxylate compound amendment to microorganisms in geologic formations according to embodiments of the invention;

FIG. 3 is a flowchart illustrating a method of measuring the effects of introduced amendments on the production of metabolic products from geologic formations according to embodiments of the invention;

FIG. 4 is a flowchart illustrating selected steps in a method of stimulating the biogenic production of compounds with enhanced hydrogen content according to embodiments of the invention;

FIG. 5 is a plot that compares methane concentrations in an unamended sample with a sample treated with an acetate amendment;

FIG. 6 is a plot showing acetate concentration over time in samples where an acetate amendment has been introduced;

FIG. 7 is a plot of methane concentration over time in an unamended sample, and samples amended with a phosphorous compound or ammonia: and

FIG. 8 is a plot of methane concentration over time in an unamended sample, and samples amended with a phosphorous compound or a mineral composition.

### DETAILED DESCRIPTION OF THE INVENTION

Methods of stimulating the production of metabolic products with enhanced hydrogen content (e.g., gases such as hydrogen and methane) through chemical amendments are described. The amendments stimulate a consortium of microorganisms in a geologic formation to metabolize carbonaceous material in the formation into the metabolic products. The stimulation effects of the amendments may include increasing the rate of production of a metabolic intermediary and/or the metabolic product. They may also include activating a consortium in the formation to start producing the metabolic products. They may further include stopping or decreasing a “rollover” effect such as when the concentration of one or more metabolic products starts to plateau after a period of monotonically increasing. These and other stimulation effects may be promoted by the chemical amendments that are introduced by the methods of the invention.

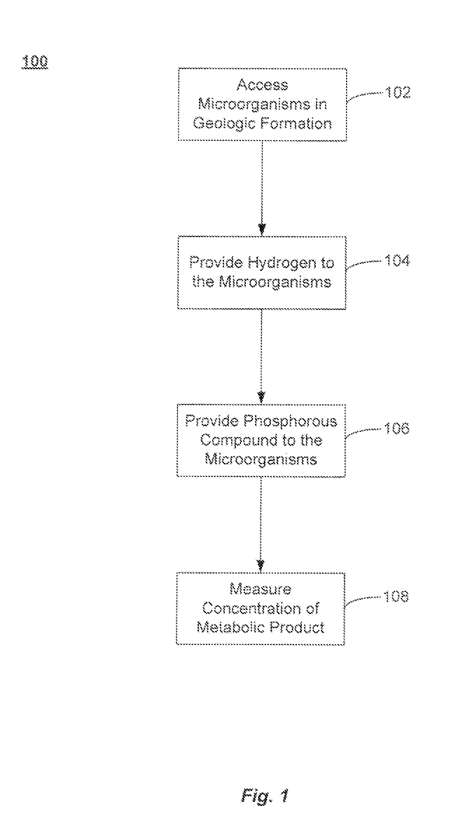

Referring now to FIG. 1, a flowchart illustrating a method 100 of introducing hydrogen and phosphorous amendments to microorganisms in a geologic formation according to embodiments of the invention is shown. The method 100 includes accessing the formation water 102 in the geologic formation. The geologic formation may be a previously explored, carbonaceous material-containing subterranean formation, such as a coal mine, oil field, natural gas deposit, carbonaceous shale, etc. In many of these instances, access to the formation can involve utilizing previously mined or drilled access points to the formation. For unexplored formations, accessing the formation may involve digging or drilling thorough a surface layer to access the underlying site where the microorganisms are located.

Once access to the microorganisms in the formation is available, an amendment may be provided to them. In method 100, providing the amendment may include providing hydrogen to the microorganisms 104. Providing the hydrogen 104 may involve the direct injection of hydrogen gas into the formation region were the microorganisms are located. Alternatively (or in addition) a liquid and/or aqueous hydrogen release compound may be provided to the formation. The compound can undergo a chemical or biochemical reaction in the formation that produces hydrogen gas in situ where the microorganisms reside. Examples of hydrogen release compounds may include polyacetate ester compounds that release lactic acid on contact with water. The lactic acid may then be metabolized by the microorganisms to produce organic acids (e.g., pyruvic acid, acetic acid, etc.) and hydrogen gas.

The amendment may also include providing one or more phosphorous compounds to the microorganisms 106. These phosphorous compounds may include phosphorous compounds (e.g., PD_{X} compounds were x is 2, 3 or 4), such as sodium phosphate (Na₃PO₄) and potassium phosphate (K₃PO₄), as well as monobasic and dibasic derivatives of these salts (e.g., KH₂PO₄, K₂HPO₄, NaH₂PO₄, Na₂HPO₄, etc.). They may also include phosphorous oxyacids and/or salts of phosphorous oxyacids. For example, the phosphorous compounds may include H₃PO₄, H₃PO₃, and H₃PO₂ phosphorous oxyacids, as well as dibasic sodium phosphate and dibasic potassium phosphate salts. The phosphorous compounds may also include alkyl phosphate compounds (e.g., a trialkyl phosphate such as triethyl phosphate), and tripoly phosphates. The phosphorous compounds may further include condensed forms of phosphoric acid, including tripolyphosphoric acid, pyrophosphoric acid, among others. They may also include the salts of condensed phosphoric acids, including alkali metal salts of tripolyphosphate (e.g., potassium or sodium tripolyphosphate), among other salts.

The hydrogen and phosphate may be provided to the formation in a single amendment, or they may be provided in separate stages. For example, if the phosphorous amendment takes the form of an aqueous solution, the solution may be injected into the formation with aid of compressed hydrogen gas. This allows the two components to be provided to the formation at substantially the same time. Alternatively, the hydrogen or phosphate amendment may be introduced first, followed by the introduction of the other compounds.

Whether the hydrogen and phosphorous compounds are introduced to the formation simultaneously or separately, they will be combined in situ and exposed to microorganisms. The combination of the hydrogen and phosphorous compound(s) can stimulate the microorganisms to metabolize carbonaceous material in the formation into metabolic products with enhanced hydrogen content, like methane. The enhanced hydrogen content products have a higher mol. % of hydrogen atoms than the starting carbonaceous material. For example, methane, which has four C—H bonds and no C—C bonds, has a higher mol. % hydrogen than a large aliphatic or aromatic hydrocarbon with a plurality of C—C single and double bonds. Additional details about compounds with enhanced hydrogen content may be found in co-assigned U.S. patent application Ser. No. 11/099,881, to Pfeiffer et al, filed Apr. 5, 2005, and entitled “GENERATION OF MATERIALS WITH ENHANCED HYDROGEN CONTENT FROM ANAEROBIC MICROBIAL CONSORTIA” the entire contents of which is herein incorporated by reference for all purposes.

Method 100 may further include adding additional amendments to the to formation. For example, a yeast extract amendment may be added to provide nutrients to the microorganisms in the formation. The yeast extract may include digests and extracts of commercially available brewers and bakers yeasts.

Method 100 may also include measuring the concentration of a metabolic product 108. For gas phase metabolic products, the partial pressure of the product in the formation may be measured, while aqueous metabolic products may involve measurements of molar concentrations. FIG. 1 shows the measurement of metabolic products being made after the introduction of the hydrogen and phosphorous amendment. Measurements may also be made before providing the amendment, and a comparison of the product concentration before and after the amendment may also be made.

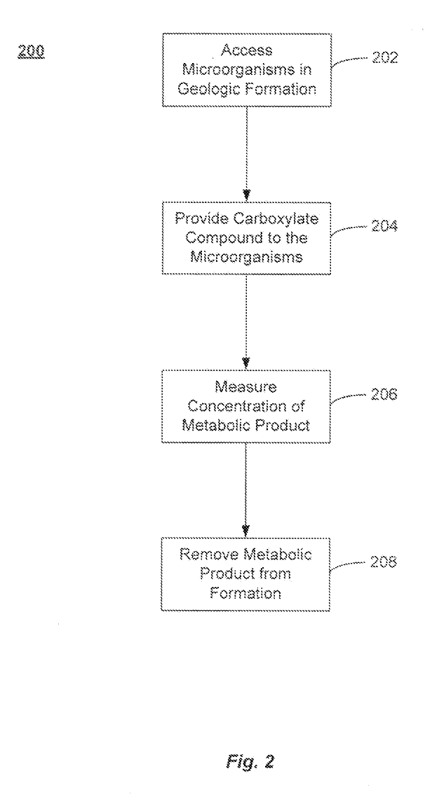

FIG. 2 shows a method 200 of introducing a carboxylate compound amendment to microorganisms in geologic formations according to embodiments of the invention. The method 200 may include accessing the microorganism in the geologic formation 202. Once access is gained, one or more carboxylate compounds may be provided to the microorganisms in situ 204. The carboxylate compound may be an organic compound having one or more carboxylate groups (e.g., COO⁻ groups). These compounds are typically organic acids or their salts. Examples include salts of acetate (i.e., H₃CCOO⁻); benzoate (i.e., Ph-COO⁻, where Ph is a phenyl group); and formate (i.e., HCOO⁻), among other carboxylate groups. Additional amendments, such as a yeast extract amendment that provides nutrients to the microorganism in the formation, may also be provided.

The concentration of a metabolic product may be measured 206 following the introduction of the carboxylate compound. The product concentration may also be measured before the carboxylate compound is introduced, to determine the effect of adding the compound. In some instances, introducing the carboxylate compound to the microorganisms may cause an almost immediate increase in the production rate of the metabolic product. In other instances, there may be a period of delay between the introduction of the carboxylate compound and an increase in the production of the metabolic product. For example, the concentration of the metabolic product in the formation may stay at pre-introduction levels for about 30, 40, 50, 60, 70, or 80 days or more before significantly increasing.

A delay of several days or months between introducing the carboxylate compound and measuring a increase in the production of the metabolic product may be called the activation period. During this time, the presence of the carboxylate compound(s) may be influencing the population or metabolic pathways of the microorganisms. Very little (or even none) of the carboxylate compound may be metabolized by the microorganisms during the activation period. In these instances, the carboxylate compound may be acting as a catalyst that activates a metabolic pathway for the production of the metabolic product. Multiple introductions of the amendment may be made over the course of the activation period to maintain a concentration level of the amendment in the formation. Alternatively, the amendment can be pulsed into the formation using discontinuous injections. Experiments demonstrating activation of methane production with an acetate amendment are described in the Experimental section below.

Method 200 may also include removing the metabolic product 208 building up in the formation as a result of the carboxylate compound amendment. If the metabolic product is a gas such as hydrogen or methane, it may be removed with conventional natural gas recovery equipment. In some examples, the products may be removed through the same access points that were used to provide the carboxylate compound to the microorganisms. In additional examples, the products may be forced out of the formation by injecting a displacement fluid (e.g., nitrogen, water, etc.) into the formation.

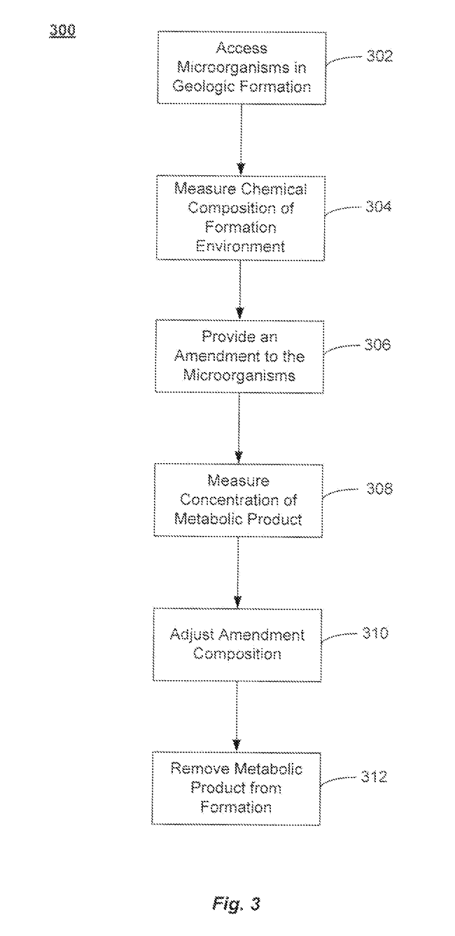

Referring now to FIG. 3, a flowchart illustrating a method 300 of measuring the effects of introduced amendments on the production of metabolic products from geologic formations is shown. The method 300 includes accessing the microorganisms 302 in a carbonaceous material containing geologic formation. Then an analysis of the microorganism formation environment may be conducted, which includes measuring the chemical composition that exists in the environment 304. This may include an in situ analysis of the chemical environment, and/or extracting gases, liquids, and solid substrates from the formation for a remote analysis.

For example, extracted formation samples may be analyzed using spectrophotometry, NMR, HPLC, gas chromatography, mass spectrometry, voltammetry, and other chemical instrumentation. The tests may be used to determine the presence and relative concentrations of elements like dissolved carbon, phosphorous, nitrogen, sulfur, magnesium, manganese, iron, calcium, zinc, tungsten, cobalt and molybdenum, among other elements. The analysis may also be used to measure quantities of polyatomic ions such as PO₂³⁻, PO₃³⁻, and PO₄³⁻, NH₄⁺, NO₂⁻, NO₃⁻, and SO₄²⁻, among other ions. The quantities of vitamins, and other nutrients may also be determined. An analysis of the pH, salinity, oxidation potential (Eh), and other chemical characteristics of the formation environment may also be performed. Additional details of chemical analyses that may be performed are described in co-assigned PCT Application No. PCT/US2005/015259, filed May 3, 2005; and U.S. patent application Ser. No. 11/343,429, filed Jan. 30, 2006, of which the entire contents of both applications are herein incorporated by reference for all purposes.

A biological analysis of the microorganisms may also be conducted. This may include a quantitative analysis of the population size determined by direct cell counting techniques, including the use of microscopy, DNA quantification, phospholipid fatty acid analysis, quantitative PCR, protein analysis, etc. The identification of the genera and/or species of one or more members of the microorganism consortium by genetic analysis may also be conducted. For example, an analysis of the DNA of the microorganisms may be done where the DNA is optionally cloned into a vector and suitable host cell to amplify the amount of DNA to facilitate detection. In some embodiments, the detecting is of all or part of ribosomal DNA (rDNA), of one or more microorganisms. Alternatively, all or part of another DNA sequence unique to a microorganism may be detected. Detection may be by use of any appropriate means known to the skilled person. Non-limiting examples include restriction fragment length polymorphism (RFLP) or terminal restriction fragment length polymorphism (TRFLP); polymerase chain reaction (PCR); DNA-DNA hybridization, such as with a probe, Southern analysis, or the use of an array, microchip, bead based array, or the like; denaturing gradient gel electrophoresis (DGGE); or DNA sequencing, including sequencing of cDNA prepared from RNA as non-limiting examples. Additional details of the biological analysis of the microorganisms is described in co-assigned U.S. patent application Ser. No. 11/099,879, filed Apr. 5, 2005, the entire contents of which is herein incorporated by reference for all purposes.

The method 300 also includes providing an amendment to the microorganisms in the formation 306. Embodiments of the present invention include providing amendments of hydrogen, phosphorous compounds, and/or carboxylate compounds (e.g., acetate) to the microorganisms. The amendments may also include vitamins, minerals, metals, yeast extracts, and other nutrients. The amendments may still further include water amendments to dilute metabolic inhibitors and/or the microorganism consortium.

The effect of the amendments can be analyzed by measuring the concentration of a metabolic intermediary or metabolic product 308 in the formation environment. If the product concentration and/or rate of product generation does not appear to be reaching a desired level, adjustments may be made to the composition of the amendment 310. For example, if an acetate amendment does not appear to be activating the microorganisms after a set period of time (e.g., 90 days or more), a different amendment may be introduced to stimulate the microorganisms (e.g., hydrogen and/or phosphorous compounds).

The method 300 may also include removing the metabolic product 312 from the formation. Removal may be triggered when the concentration of the reaction product increases above a threshold level in the formation. In some of these instances, removal may performed to keep the product in a concentration range that has been found to stimulate the microorganisms to generate more of the product.

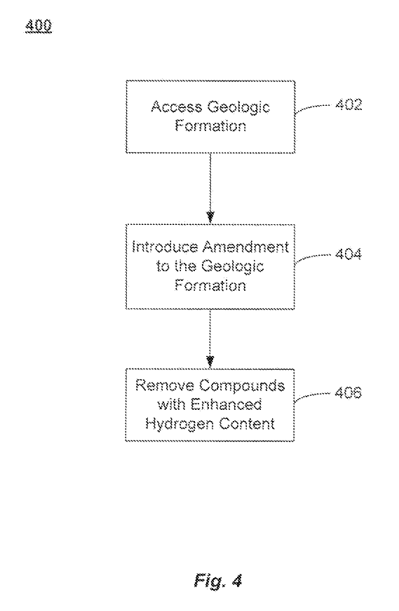

FIG. 4 shows a method 400 of introducing an amendment to a geologic formation to stimulate the biogenic production of compounds with enhanced hydrogen content according to embodiments of the invention. The method may include accessing a geologic formation that contains a carbonaceous material 402. The method may further include providing the amendment to the geologic formation 404. The method may still further include removing the compounds with the enhanced hydrogen content from the formation 406.

The amendments added to the geologic formation is step 404 may include, without limitation, salts of alkali metals, such as salts of potassium. Specific examples may include alkali metal halide salts such as sodium chloride, potassium chloride, sodium bromide, potassium bromide, sodium iodide, and potassium iodide, among others. They may also include alkali metal phosphate salts, such as sodium phosphate, potassium phosphate, and sodium triphosphate, among others. The amendments may also include salts of alkali earth metals, such as salts of calcium and magnesium. These salts may include halide salts of alkali earth metals such as calcium chloride (CaCl₂) and magnesium chloride (MgCl₂). The salts may further include alkali earth metal phosphate salts such as calcium phosphate and magnesium phosphate.

Additional examples of the amendments may include, without limitation, vitamin amendments such as vitamin B-12, niacin, thiamin, riboflavin, biotin, pantothenic acid, and folate, among others.

Still more examples of the amendments may include proteins and materials that containing protein including, without limitation, milk proteins, hydrolyzates of milk proteins such as casein hydrolyzates, soy protein, peptones, yeast extract, and Brewer's yeast, among others.

Examples of the amendments may further include organic acids such as, without limitation, formic acid, acetic acid, propionic acid, butyric acid, lactic acid, and decanoic acid, among others. Examples of amendments may also include salts of organic acids, such as sodium, potassium, calcium, and magnesium salts of the above-described organic acids.

Examples of the amendments may still further include glycerol, glyceryl triacetate, ethyl lactate, and polyoxyethylene, among other compounds.

Examples of amendments may further include tracer compounds that provide evidence about the extent to which an amendment has been diluted in the geologic formation.

For example, a tracer compound having a first concentration may be injected into the formation and then at least a portion of the tracer compound may be pulled out having a second concentration. The decrease in concentration between the first and second concentration provides evidence of the extent the amendment included the tracer was diluted in the formation. The dilution may occur when the added amendment is mixed with water or other liquids already present in the formation. Specific examples of tracer compounds may include halogen salts (e.g., chloride, bromide and/or iodide salts) such as alkali metal and/or alkali-earth metal bromides, and alkali metal and/or alkali-earth metal iodides.

In additional embodiments, removal of the metabolic product may be done independently of the product concentration in the formation. For example, the reaction products may be continuously removed from the formation as part of a process that cycles the amendment through the formation. The mixture of metabolic products, amendment components and other materials removed from the formation may be processed to separate the products from components that will be sent back into the formation.

### EXPERIMENTAL

### Hydrogen and Phosphorus Compound Amendments

Experiments were conducted to compare biogenic methane generation from coal samples after introducing an amendment of hydrogen gas, a phosphorous compound, and ammonia. For each experiment, methane generation from coal samples from the Monarch coal seam in the Powder River Basin in Wyoming was periodically measured over the course of about 627 days. Each 5 gram coal sample was placed in a 30 ml serum bottle with 15 mL of water that was also taken from the formation. The coal and formation water were placed in the serum bottle while working in an anaerobic glove bag. The headspace in the bottle above the sample was flushed with a mixture of N₂ and CO₂ (95/5).

Amendments were then added to the samples. In a second set of experiments, 4.5 mL of H₂ gas (i.e., 179 μmmol of H₂) was added to each bottle. Also added to the bottles was 0.15 mL of a 2500 mg/L (as N) aqueous ammonium chloride solution to provide a concentration of 25 mg/L, as nitrogen, to the samples, and 0.04 mL of a 1800 mg/L potassium phosphate solution that provided a concentration of 5 mg/L, as phosphate, to the samples. In a second set of experiments, the same amount of H₂ was added to the bottles, but no ammonium chloride or potassium phosphate. A third set of experiments introduced the ammonium chloride and potassium phosphate at the same levels as the first set, but no hydrogen gas was added. The samples were then sealed, removed from the glove box, and stored at room temperature over the course of the experiments.

The methane levels in the headspace above the samples was periodically measured and recorded. The methane was measured by running samples of the headspace gases through a gas chromatograph equipped with a thermal conductivity detector. The highest levels of methane production after 627 days occurred in samples treated with an amendment of hydrogen gas, ammonium chloride, and potassium phosphate, with average levels reaching 248 μmmol of CH₄. This compares with 128 μmmol CH₄ for samples just having the H₂ amendment, and 64 μmmol CH₄ for samples just having the ammonia and phosphorous compound amendment.

The combination of the hydrogen and potassium phosphate generated more methane than can be accounted for by methanogenic conversion of the added hydrogen to methane. In the methanogenic metabolism of hydrogen to methane, four moles of molecular hydrogen and 1 mole of carbon dioxide are converted into 1 mole of methane:

4H₂+CO₂→CH₄+2H₂O

This means the 179 μmmols of H₂ added to the sample bottles could, at most, be converted into 44.7 μmmols of methane. For samples measuring peak methane production of 248 μmmols, this leaves 203 μmmols coming from other sources. Samples without hydrogen amendments produced about 63 μmmols of methane from these coal substrates. This still leaves at least 185 μmmols of methane that was generated from another source.

The source of the additional methane is believed to come from the biogenic metabolism of the coal into methane. The hydrogen and phosphorous compound amendment is believed to have stimulated the microorganisms present in the sample to metabolize the coal into methane. The stimulatory effect of the hydrogen and phosphorous amendment is not limited to enhancing the conversion of the added hydrogen gas to methane. It also includes stimulating the microorganisms to use methanogenic metabolic pathways that convert the coal substrate into methane.

### Acetate Amendments

Experiments were conducted to measure the effects of acetate amendments on methane production from samples of carbonaceous materials. The carbonaceous materials used in these experiments were coal samples taken from underground coal beds at the Monarch coal site. The samples were transported under anaerobic conditions to 30 ml serum bottles, where 1 gram samples of the coal were combined in an anaerobic glove box with 20 mL of formation water from the same site and 0.2 mL of cell concentrate. The cell concentrate consisted of cells from about 6.6 L of formation water added to 15 mL of formation water. The headspace in the bottle above the sample was exchanged with a mixture of N₂ and CO₂ (95/5).

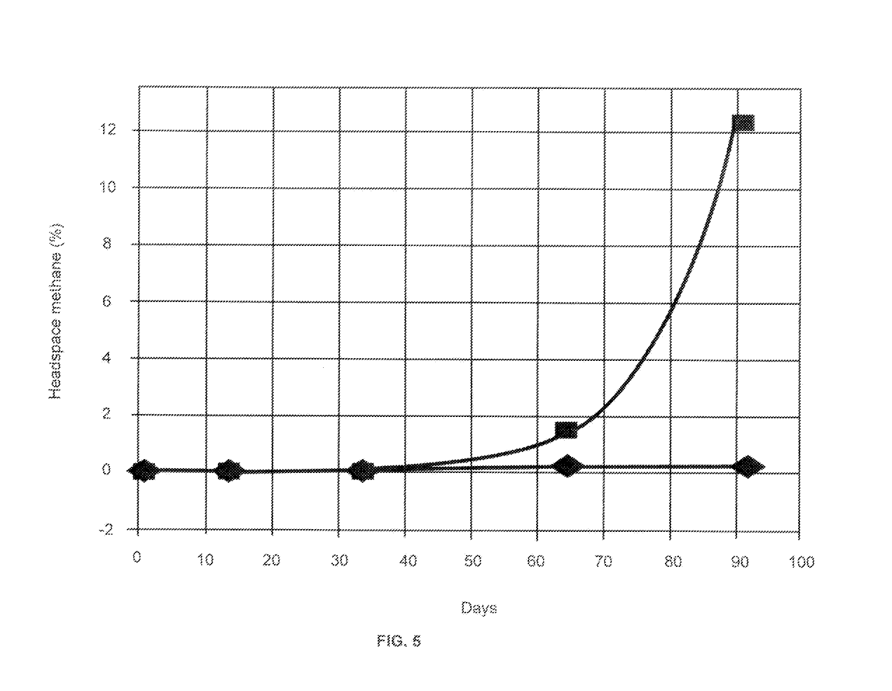

In a first set of samples, the acetate amendment included adding an aqueous sodium acetate solution to the sample bottles to give the samples a 10 mg/mL acetate concentration. A second set of control samples were prepared in the same manner except for lacking the acetate amendment. Methane levels (measured as a mol. % methane in the headspace of the sample bottle) were periodically measured in both the amendment and control samples over the course of 90 days. FIG. 5 shows a plot of the methane levels measured in these samples as a function of time.

As FIG. 5 reveals, very little methane generation occurred in either the amendment or control sample during the first 50 days. But the measurement taken on day 65 shows the methane levels starting to build in the acetate amendment sample while the control sample continued to show negligible methane generation. By the 90th day, the acetate amendment sample showed rapid and significant methane generation with methane representing over 12 mol. % of the headspace in the sample bottles. Meanwhile, the control samples that lacked the acetate amendment still showed almost no methane generation after 90 days.

Plot of FIG. 5 clearly shows that the acetate amendment had a significant impact on methane generation after an activation period of about 65 days. But the plot did not show whether the methane was produced by the methanogenic conversion of the acetate into methane, or whether the methane was derived from the coal sample. Thus, a second measurement was made of the acetate concentration in the samples over the same period of time.

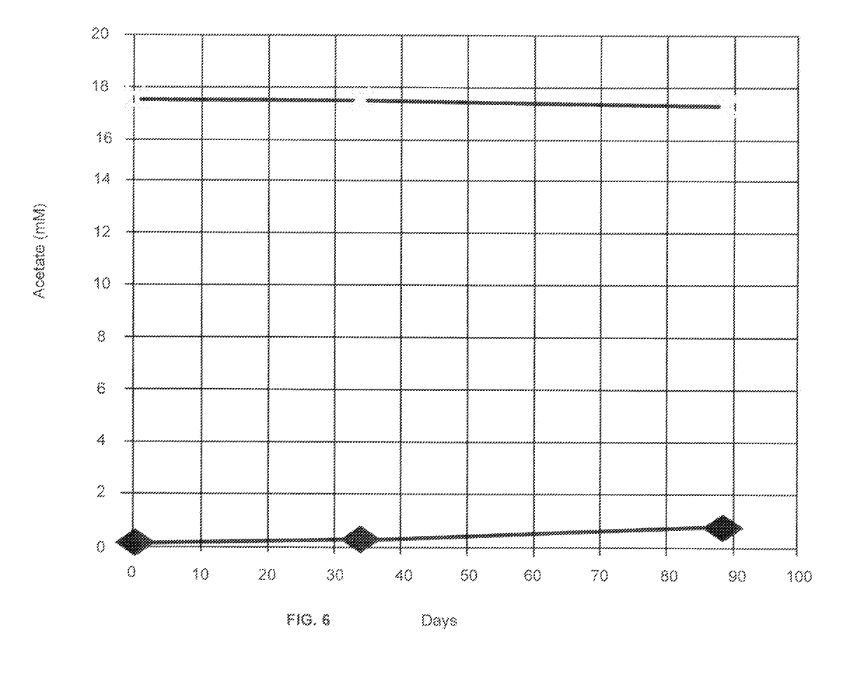

FIG. 6 shows the plot of the acetate concentrations over time in the samples. The plot reveals that the acetate concentration did not change significantly over the 90 day period. Most significantly, little change in the acetate concentration was observed before and after the methane generation rapidly increased in the acetate amendment samples. These data indicate that the acetate amendment acted as an activation agent to enhance the methanogenic metabolism of the coal into methane. The data also show that the acetate activation does not occur immediately, and that a delay of several weeks to months may occur before the start of significant methanogenic activity.

### Phosphorous Compound Amendments and Rollover

Rollover is a condition where the rate of biogenic methane production starts to plateau as the in situ methane concentration reaches a certain level. In many instances, the rate flattens to zero, and the methane concentration remains constant over time. The rollover point (i.e., the point where the methane concentration begins to break from a monotonically increasing state) can vary between microorganism consortia, but appears to be reached in almost all unamended samples of carbonaceous material that have been examined to date.

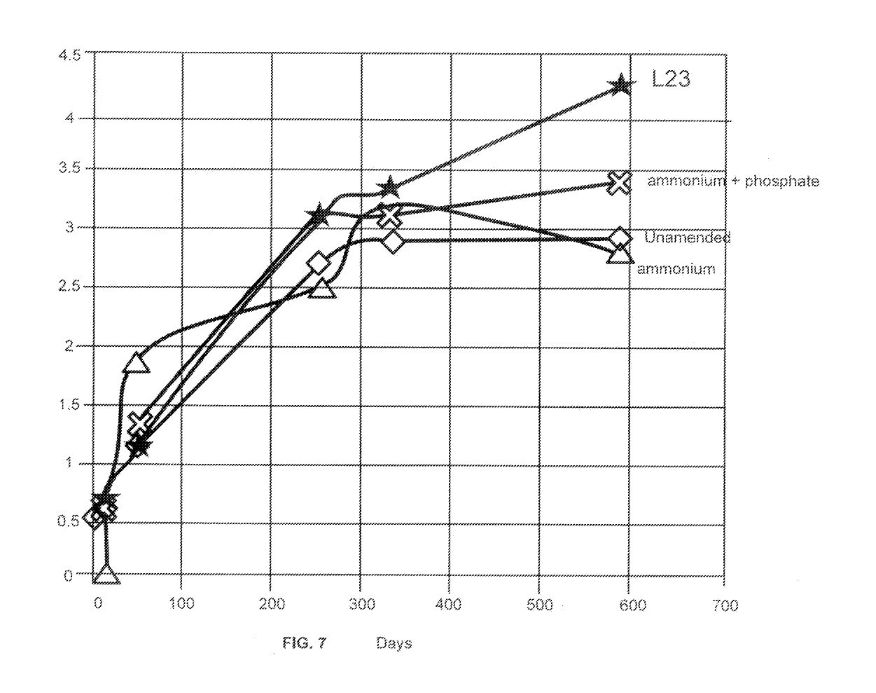

But some samples receiving minerals, metals and nutrient amendments exhibited less of a rollover effect than unamended controls. Further tests revealed that the agents responsible for reducing rollover were phosphate compounds, such as sodium or potassium phosphate. FIG. 7 shows a plot of methane levels over time in the headspace of 30 ml serum bottles containing amended and unamended coal samples. The plot for the unamended sample shows the rollover point occurring when the methane level in the headspace reaches between 2.5 and 3 mol. %. At these methane levels, the rate of methane production starts to decrease and the methane level remains constant at slightly under 3 mol. %.

A more volatile, but similar pattern was observed for samples treated with an ammonium amendment. In these samples, ammonium chloride was introduced to give each sample a concentration of 25 mg/L nitrogen at the start of the methane measurements. The rate of methane production in these samples was initially greater than for the unamended samples or samples with other types of amendments (including an amendment of ammonium and phosphate). In addition, the peak methane level in the ammonium samples exceeded the peak plateau levels in the unamended samples. But eventually the methane levels began to decrease, and by about day 600 the methane levels in the samples were about the same as those measured in the unamended samples.

The samples treated with an amendment that included a phosphorous compound (i.e., potassium phosphate) all appeared to breakthrough the plateau methane level observed in the samples that were prone to rollover. As FIG. 7 shows, samples treated with a pure 5 mg/L potassium phosphate amendment had a methane level of about 4.3 mol. % after 600 days, or about 43% higher than samples without phosphate. Amendments with ammonium chloride and phosphate did not result in substantial increases.

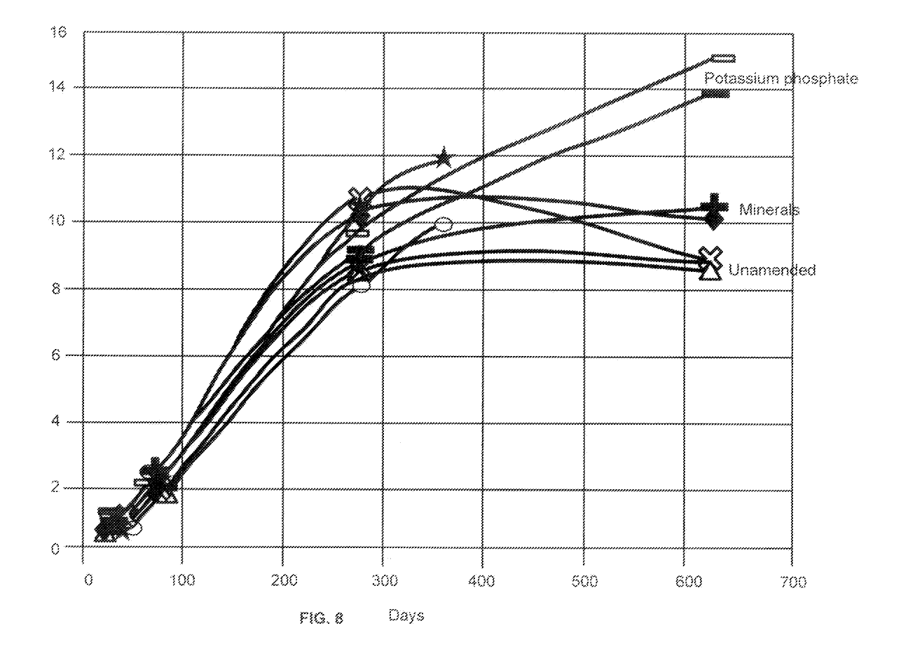

FIG. 8 shows another plot of methane concentration over time for samples with and without phosphorous compound amendments. Similar to the plot in FIG. 7, this plot shows samples that were not treated with a phosphorous amendment (i.e., a potassium phosphate amendment) reached a rollover point beyond which the methane concentration did not increase. In contrast, no plateau was observed in the methane concentration of two sets of samples that were treated with a phosphate amendment. At the end of just over 600 days, the phosphate containing samples had significantly higher methane levels than samples treated with a minerals amendment or the samples that were unamended.

FIGS. 7 and 8 indicate that phosphorous compounds such as potassium phosphate can extend methanogenesis supported by complex hydrocarbons. Thus, the introduction of a phosphorous compound amendment to microorganisms in a geologic formation may stimulate the microorganisms to continue to produce methane in an environment where they are already exposed to high levels of methane.

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the microorganism” includes reference to one or more microorganisms and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A method of stimulating biogenic production of a metabolic product with enhanced hydrogen content, the method comprising:
accessing a geologic formation that includes a carbonaceous material;
introducing an amendment to the geologic formation; and
removing the metabolic products with enhanced hydrogen content from the formation.

2. The method of claim 1, wherein the amendment comprises at least one alkali metal compound or alkali earth metal compound.

3. The method of claim 2, wherein the alkali metal compound or alkali earth metal compound comprises a halide salt.

4. The method of claim 3, wherein the alkali metal halide salt or alkali-earth metal halide salt comprises sodium chloride, potassium chloride, sodium bromide, potassium bromide, sodium iodide, potassium iodide, calcium chloride, or magnesium chloride.

5. The method of claim 2, wherein the alkali metal compound or alkali earth metal compound comprises a phosphate salt.

6. The method of claim 5, wherein the alkali metal phosphate or alkali-earth metal phosphate comprises sodium phosphate, potassium phosphate, sodium triphosphate, calcium phosphate, or magnesium phosphate.

7. The method of claim 1, wherein the amendment comprises at least one vitamin.

8. The method of claim 7, wherein the vitamin comprises vitamin B-12, niacin, thiamin, riboflavin, biotin, pantothenic acid, or folate.

9. The method of claim 1, wherein the amendment includes at least one protein or material containing protein.

10. The method of claim 9, wherein the protein or material containing protein comprises milk proteins, casein hydrolyzates, soy protein, peptones, yeast extract, or Brewer's yeast.

11. The method of claim 1, wherein the amendment comprises at least one organic acid.

12. The method of claim 11, wherein the organic acid comprises formic acid, acetic acid, propionic acid, butyric acid, lactic acid, or decanoic acid.

13. The method of claim 1, wherein the amendment comprises an alkali metal or alkali earth metal salt of an organic acid.

14. The method of claim 13, wherein the alkali metal or alkali earth metal salt of an organic acid comprises a sodium, potassium, calcium, or magnesium salt of the organic acid.

15. The method of claim 1, wherein the amendment comprises at least one of glycerol, glyceryl triacetate, ethyl lactate, or polyoxyethylene.

16. The method of claim 1, wherein the amendment comprises a tracer compound.

17. The method of claim 1, wherein carbonaceous material comprises coal, oil, carbonaceous shale, oil shale, tar sands, tar, lignite, kerogen, bitumen, or peat.

18. The method of claim 1, wherein the metabolic products with enhanced hydrogen content comprise methane or hydrogen.
